SEQUENCE LISTING (2 sequences)

SEQ ID NO: 1 - DNA, 20 bp, Artificial
  artificially created hBCRP1For primer
ccatagcagcaggtcagagt

SEQ ID NO: 2 - DNA, 20 bp, Artificial
  artificially created hBCRP1-Rev primer
aggccacgtgattcttccac

(19) United States
(12) Patent Application Publication
(10) Pub. No.: US 2012/0052005 A1
(43) Pub. Date: Mar. 1, 2012

(54) COMBINATION THERAPY TO IMPROVE DRUG EFFICIENCY

(75) Inventors: Rui Ding, Edison, NJ (US); Jia Shi, Columbus, NJ (US); Kathleen W. Scotto, Washington Crossing, PA (US)

(73) Assignee: University of Medicine and Dentistry of New Jersey, Somerset, NJ (US)

(21) Appl. No.: 13/214,887

(22) Filed: Aug. 22, 2011

Related U.S. Application Data
(63) Continuation-in-part of application No. PCT/US2010/024847, filed on Feb. 20, 2010.
Application No. PCT/US2010/024847 is a continuation-in-part of application No. 13/202,377.
(60) Provisional application No. 61/208,138, filed on Feb. 20, 2009.

(51) Int. Cl. A61K 51/00; A61K 49/00; A61P 29/00; A61P 33/10; A61P 9/06; A61P 31/00; A61P 7/02; A61P 25/24; A61P 3/10; A61P 25/08; A61P 37/08; A61P 9/14; A61P 31/04; A61P 35/00; A61P 37/06; A61P 5/14; A61P 31/12; A61P 25/22; A61K 31/56; A61P 7/04; A61P 21/00; A61P 5/24; A61P 9/08; A61P 35/02; A61K 31/522

(52) U.S. Cl. 424/1.11; 514/263.34; 424/9.1; 514/171

(57) ABSTRACT

Compositions and methods for increasing drug bioavailability and/or preventing multi-drug resistance through inhibition of ABCG2 by xanthine compounds are disclosed.

### CROSS-REFERENCE TO RELATED APPLICATIONS

This application is a continuation-in-part of International Application No. PCT/US2010/024847, filed Feb. 20, 2010, and its U.S. national phase application Ser. No. 13/202,377, filed Aug. 19, 2011, both of which claim priority to U.S. Provisional Application No. 61/208,138, filed Feb. 20, 2009. The disclosures of the above-described prior applications are incorporated herein by reference in their entirety for all purposes.

### FIELD OF THE INVENTION

The instant invention is related to improvement of drug efficiency by increasing their bioavailability and/or reversing or preventing drug resistance with xanthine compounds, such as caffeine and caffeine analogs.

### BACKGROUND OF THE INVENTION

Multi-drug resistance (MDR) in tumor cells is a significant obstacle to the success of chemotherapy in many cancers. Multidrug resistance is a phenomenon whereby tumor cells in vitro that have been exposed to one cytotoxic agent develop cross-resistance to a range of structurally and functionally unrelated compounds. The drug resistance that develops in cancer cells often results from elevated expression of particular proteins, such as cell-membrane transporters, which can result in an increased efflux of the cytotoxic drugs from the cancer cells, thus lowering their intracellular concentrations.

In addition, MDR occurs intrinsically in some cancers without previous exposure to chemotherapy agents. The cytotoxic drugs that are most frequently associated with MDR are hydrophobic, amphipathic natural products, such as the taxanes (paclitaxel, docetaxel), vinca alkaloids (vinorelbine, vincristine, vinblastine), anthracyclines (doxorubicin, daunorubicin, epirubicin), epipodophyllo-toxins (etoposide, teniposide), topotecan, dactinomycin, and mitomycin C.

Although MDR can have several causes, one major mechanism of resistance to chemotherapy involves ABC transporters, these transporters can efflux the hydrophobic drugs against osmotic pressure. Members involved in the drug resistance include p-glycoprotein MRP1 and ABCG2.

ABCG2 is an ATP-binding-cassette (ABC) trans-membrane protein that was first identified by virtue of its over-expression in breast cancer cells, thereby it is also known as Breast Cancer Resistance Protein (BCRP). The over-expression of ABCG2 has been observed in breast cancer cells as well as in other cancer types. Additionally, ABCG2 has been found overexpressed in certain stem cell populations, contributing to stem cell state maintenance. It has been demonstrated that ABCG2 confers drug resistance to chemo-therapeutic reagents, such as mitoxantrone, topotecan, and some of the most recent developed anticancer drug SN38, as well as other toxins and carcinogens in food products and endogenous compounds.

In normal tissues, ABCG2 is found in the epithelium of the small intestine, the ducts, the vascular endothelium, and liver canalicular membranes. It is believed that ABCG2 plays an important role in absorption, distribution, and excretion of xenobiotics, which may restrict bio-availability of ABCG2 substrates when administered drugs fall into this category. Since ABCG2 is a transmembrane protein on cancer cells, direct resistance to the chemo-drugs exists regardless of the administration method of the drugs. Therefore, ABCG2 function inhibition and/or gene expression down-regulation has been proposed as part of the remedy to improve therapeutic efficacy.

To date, considerable efforts have been made to understand the molecular mechanisms of ABCG2 gene expression regulation as it relates to multi-drug resistance for the development of effective therapeutic strategies. However, the understanding of ABCG2 mechanism of action is far from comprehensive and there remains a need for inhibitors of ABCG2 useful for reducing multi-drug resistance and/or increasing drug bioavailability.

### SUMMARY OF THE INVENTION

This need is met by the present invention. It has now been discovered that xanthine compounds such as caffeine and its analogs antagonize ABCG2 expression. Because ABCG2 has been demonstrated to confer multi-drug resistance in tumor cells and restrict bioavailability in other tissues in addition to tumor cells, xanthine compounds can be used to sensitize ABCG2-expressing tumor cells to chemotherapeutic agents and also to increase the bioavailability of drugs in general, including chemotherapeutic agents.

Therefore, in one aspect of the present invention, a pharmaceutical composition is provided, combining a pharmaceutically active agent that is an ABCG2 substrate and a xanthine compound that is present in an amount effective to increase the oral bioavailability of the pharmaceutically active agent, wherein the xanthine compound has a structure according to formula II:

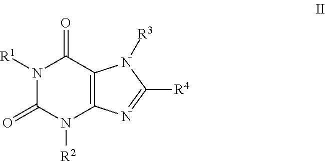

wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen, or C₁-C₆ alkyl optionally substituted by one to three substituents independently selected from hydroxyl and halogen; and
R⁴ is selected from hydrogen, C₁-C₆ alkyl, C₃-C₆ cycloalkyl, C2-C₆ alkenyl, C₂-C₆ alkynyl, aryl, arylalkyl, and arylalkenyl, wherein the aryl or aryl part of the arylalkyl and arylalkenyl is optionally substituted by one to five substituents independently selected from the group consisting of halogen, C₁-C₆ alkyl, hydroxyl and C₁-C₆ alkoxy.

In one embodiment of this aspect, the pharmaceutical composition comprises a chemotherapeutic agent. In another embodiment, the pharmaceutical composition comprises an amount of the xanthine compound of Formula (II) effective to prevent the tumor from developing resistance to the chemo-therapeutic agent. Examples of the xanthine compounds suitable for the present invention include, but are not limited to, caffeine and analogs that will be described in more detail below.

In another aspect the present invention provides a method of treating a patient having a disease or condition associated with expression of ABCG2, comprising administering to the patient a therapeutically effective amount of a composition comprising a pharmaceutically active agent and a xanthine compound, wherein the pharmaceutically active agent is an ABCG2 substrate and the xanthine compound has a structure according to formula II.

In one embodiment of this aspect, the method is provided for the treatment of ABCG2-expressing tumor cells in a patient being treated with a chemotherapy drug that is an ABCG2 substrate, in which a xanthine compound of Formula II is administered to the patient in combination with the chemotherapy drug in an amount effective to increase the efficacy of a chemotherapy drug against the tumor cells, or to prevent the tumor cells from developing resistance to the chemotherapy drug, or both.

The xanthine compound and the chemotherapy drug may be administered by multiple routes including, without limitations, oral administration, intravenous administration, intraperitoneal (IP) administration, intraarterial administration, intra-muscular administration, intracolonic administration, intracranial administration, intra-thecal administration, intra-ventricular administration, intraurethral administration, intra-vaginal administration, subcutaneous administration, intraocular administration, intranasal administration, or any combinations thereof. The xanthine compound is administered prior to, simultaneously with, or after the administration of the pharmaceutically active agent.

In another aspect the present invention provides a method of improving bioavailability of a pharmaceutically active agent delivered across an ABCG2 expressing membrane to a patient in need thereof, comprising administering to the patient the pharmaceutically active agent in combination with a xanthine compound according to formula II, wherein the pharmaceutically active agent is an ABCG2 substrate.

As in the previous aspect, the xanthine compound is administered prior to, simultaneously with, or after the administration of the pharmaceutically active agent. In this aspect, it is preferred that both the xanthine compound and the pharmaceutically active agent are administered via the same route.

In certain embodiments, the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, phenylpropanolamine. In other embodiments, the composition is not coffee, tea, or a caffeinated soft beverage.

The xanthine compound may, in different embodiments, be selected from theophylline, pentoxifylline, iso-caffeine, 8-cyclopentyl-1,3-dipropylxanthine (DPCPX), 3,7-dimethyl-1-propargylxanthine (DMPX), and 8-(3-chlorostyryl)caffeine (CSC), or the like.

The instant invention also provides for a use of a composition comprising a xanthine compound according to formula II for the manufacture of a medicament with increased bioavailability of an active pharmaceutical agent that is an ABCG2 substrate. In one embodiment, the pharmaceutically active agent is a chemotherapy drug.

Thus, the compositions of the instant invention are used for manufacture of a medicament for treatment of a cancer, which, in some embodiments, may be a multi-drug resistant cancer. In yet another embodiment, the pharmaceutically active agent is a nonchemotherapy substrate of ABCG2.

These and other aspects of the present invention will be better appreciated by reference to the following drawings and detailed description.

### BRIEF DESCRIPTION OF THE DRAWINGS

FIGS. 1A-1E illustrate the decrease of the protein level of ABCG2 and reversibility of that decrease.

FIG. 2 illustrates the effect of caffeine on cellular distribution of ABCG2 protein.

FIGS. 3A and 3B illustrate lack of effect of caffeine on ABCG2 mRNA level.

FIG. 4 illustrates increase of intracellular retention of an ABCG2 specific substrate by caffeine.

FIGS. 5A-5G illustrate that Caffeine increases the apoptotic population of Bewo cells in the mitoxantrone treatment.

FIGS. 6A-6C illustrate the effects of different caffeine analogs on the level of ABCG2 protein.

FIG. 7 illustrates the effect of PI3K inhibitor LY294002 on the level of ABCG2 protein.

FIGS. 8A and 8B illustrate the effect of adenosine receptor (AR) antagonists DPCPX and DMPX on the level of ABCG2 protein.

FIG. 9 illustrates that AR antagonists, namely caffeine and caffeine analogs CSC, DMPX, and DPSCZ, decrease the level of ABCG2 protein.

FIGS. 10A and 10B illustrate that adenosine reverses caffeine-mediated downregulation of ABCG2 protein.

FIG. 11 illustrates that adenosine kinase inhibition reverses the downregulation of ABCG2 by caffeine. Adenosine phosphorylation is required for xanthines to downregulate ABCG2.

FIG. 12 illustrates that nucleoside transporter inhibition prevented adenosine from reversing the effect of caffeine. Adenosine must be transported into the cell to prevent caffeine from downregulating ABCG2.

FIG. 13 illustrates that only adenosine receptor antagonists that are xanthines decrease ABCG2 protein.

FIG. 14 illustrates adenosine mediated signaling pathways.

### DETAILED DESCRIPTION OF THE INVENTION

The present invention is based on a surprising discovery that xanthine compounds such as caffeine and analogs thereof decrease the amount of ABCG2 expressed by cancer cells and healthy tissues and alter its distribution. Further, the inventors have surprisingly discovered that as a consequence the xanthine compounds increase the sensitivity of cancer cells to chemotherapy drugs.

Accordingly, the instant invention is drawn to various aspects stemming from these discoveries. Among others, the disclosure provides that xanthines downregulate ABCG2 expressing cells to chemotherapeutic agents, that xanthines downregulate ABCG2 by inducing its lysosomal degradation, and that adenosine-mediated intracellular events are involved in this regulation. In particular, two xanthine derivatives, DMPX and DPCPX, reduced ABCG2 protein levels at pharmaceutically relevant concentrations, and DPCPX is one of the most active compounds tested so far.

The present invention is particularly useful in providing compositions and methods for increasing sensitivity of cancer cells to chemotherapeutic drugs or enhancing bioavailability of active pharmaceutical ingredients in general. The compositions comprise a xanthine compound such as caffeine or an analog thereof.

Caffeine is a 1,3,7-trimethyxanthine with a purine-like structure that it is highly permeable to cell membrane. A variety of pharmacological effects of caffeine have been described, most dominant of which contributes to central nervous system stimuli via inhibition of adenosine receptors. Given the wide presence in a variety of dietary supplies, caffeine has become the most highly consumed psychoactive substance in the world. Besides, caffeine is also applied therapeutically in many ways, such as the treatment of migraines, respiratory stimulation in neonates, radio-sensitization, postprandial hypotension and obesity.

The structure of caffeine is well known and is illustrated in Formula I below:

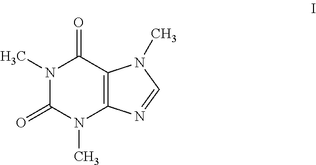

In one aspect the present invention provides a pharmaceutical composition comprising a pharmaceutically active agent and a xanthine compound, wherein the pharmaceutically active agent is an ABCG2 substrate and the xanthine compound has a structure according to formula II:

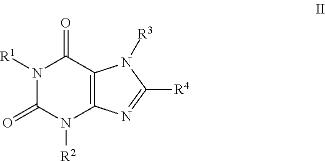

wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen, or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, alkenyl, C₂-C₆ alkynyl, aryl, arylalkyl, and arylalkenyl, wherein the alkyl, cycloalkyl, and aryl or aryl part of the arylalkyl and arylalkenyl is optionally substituted by one to five substituents independently selected from the group consisting of halogen, C₁-C₆ alkyl, hydroxyl and C₁-C₆ alkoxy.

In one embodiment of this aspect, the xanthin compound is characterized by Formula II as described above, wherein R¹, R², R³ and R⁴ are not all concurrently hydrogen.

In another embodiment of this aspect, the pharmaceutically active agent is not ergotamine tartrate, acetaminophen, ibuprophen, Isometheptene Mucate, acetylsalicylic acid or a salt thereof, butalbital, Propoxyphent, Pyrilamine maleate, chlorpheniramine, or phenylpropanolamine.

In another embodiment of this aspect, the composition is not coffee, tea, or a caffeinated soft beverage or energy beverage.

In another embodiment of this aspect, the xanthin compound is characterized by Formula II as described above, wherein R¹, R², R³ and R⁴ are not all concurrently hydrogen; the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, phenylpropanolamine; and the composition is not coffee, tea, or a caffeinated soft beverage or energy beverage.

In another embodiment of this aspect, the xanthine compound has a structure of Formula II, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl, preferably, wherein R¹, R², R³ and R⁴ are not all concurrently hydrogen.

Some active metabolites of these xanthine compounds may also be called “caffeine analog(s).” In a preferred embodiment, the xanthine compounds of the present invention are selected from the group consisting of the caffeine analogs listed in the following Table.

TABLE
Caffeine Analogs of Formula (II)

| Compound | Compound | | | | |
|---|---|---|---|---|---|
| No. | Name | R¹ | R² | R³ | R⁴ |
| 1 | Caffeine | CH₃ | CH₃ | CH₃ | H |
| 2 | Theophylline | CH₃ | CH₃ | H | H |
| 3 | Paraxanthine | CH₃ | H | CH₃ | H |
| 4 | Theobromine | H | CH₃ | CH₃ | H |
| 5 | Dyphylline | CH₃ | CH₃ | CH₂CH(OH)CH₂OH | H |
| 6 | DPCPX | n-Pr | n-Pr | H | cyclopentyl |
| 7 | DMPX | HC≡CCH₂— | CH₃ | CH₃ | H |
| 8 | CSC | CH₃ | CH₃ | CH₃ | 3-Cl-styryl |

The structures of these caffeine analogs are specifically listed below.

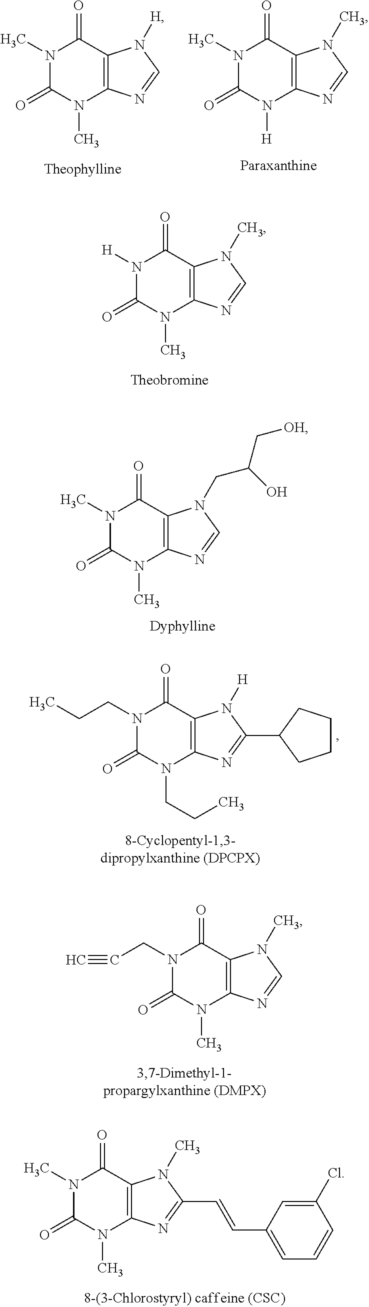

In one embodiment of this aspect, the caffeine analog is not Dyphylline, 7-(β-Hydroxyethyl)theophylline, Paraxanthine, or 7-methylxanthine.

In a preferred embodiment, the caffeine analog is selected from the group consisting of theophylline, pentoxyphyline and iso-caffeine. In another preferred embodiment, the caffeine analog is 8-cyclopentyl-1,3-dipropylxanthine (DPCPX). In another preferred embodiment, the caffeine analog is 3,7-dimethyl-1-propargylxanthine (DMPX). In another preferred embodiment, the caffeine analog is 8-(3-chlorostyryl)caffeine (CSC).

The composition can be administered orally, intravenously, intraarterially, intramuscularly, intracolonically, intracranially, intrathecally, intraventricularly, intra-urethrally, intravaginally, subcutaneously, intraocularly, intranasally, topically, or by any combinations thereof. In a preferred embodiment, the pharmaceutically active agent is suitable for oral administration.

In another embodiment of this aspect, the pharmaceutically active agent is selected from analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineo-plastic agents, immunosuppressants, antithyroid agents, antiviral agents, anxiolytic sedatives, astringents, beta-adrenoceptor blocking agents, calcium channel blockers, contrast media, corticosteroids, cough suppressants, diagnostic agents, diagnostic imaging agents, diuretics, dopamin-ergics, endogenous substances, haemostatics, immuriological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators, and any other agents that arc substrates of ABCG2.

In another embodiment of this aspect, the pharmaceutically active agent is a chemotherapy drug, which include, without limitations, topoisomerase I inhibitors, such as NB-506, edotecarin (J-10788), and becatecarin; topoisomerase II inhibitors, such as etoposide, teniposide, and various camptothecin derivatives, such as topotecan, irinotecan (CPT-11), SN-38, diflomotecan (BN80915), 9-aminocamptothecin, karenitecin (BNP 1350), gimatecan, and exatecan (DX-891f); mitoxantrone, bisantrene, anthracyclins, such as daunorubicin, doxorubicin, epirubicin; methotrexate; and tyrosine kinase inhibitors, such as gefitinib, imatinib, carnetinib (CI033), nilotinib, desatinib, sunitinib, and erlotinib. In a preferred embodiment, the chemotherapy drug is selected from mitoxantrone, topotecan, SN38, and analogs thereof.

Other chemotherapy or nonchemotherapy agents that have been or are to be identified as ABCG2 substrates are also encompassed by the present invention. For a review of chemotherapy agents or nonchemotherapy agents that are ABCG2 substrates, see H. E. M. zu Schwabedissen and H. K. Kroemer, “In Vitro and In Vivo Evidence for the Importance of Breast Cancer Resistance Protein Transporters (BCRP/MXR/ABCP/ABCG2),” in Drug Transporters, Handbook Experimental Pharmacology 201, M. F. Fromm and R. B. Kim eds., Springer-Verlag Berling Heidelberg (2011), which is hereby incorporated by reference in its entirety.

In another embodiment, the pharmaceutically active agent is an inhibitor of at least one protein associated with development of multi-drug resistance. In a preferred embodiment, the at least one protein associated with development of multi-drug resistance is a P-glycoprotein, multidrug resistance-associated protein, or lung resistance-related protein.

In another embodiment of this aspect, the composition further comprises a chemotherapy drug.

In another embodiment of this aspect, the one protein is a P-glycoprotein; and the xanthine compound has a structure characterized by formula II, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl, preferably, wherein R¹, R², R³ and R⁴ are not all concurrently hydrogen.

In a preferred embodiment, the xanthine compound is selected from the group consisting of theobromine, 8-cyclopentyl-1,3-dipropylxanthine (DPCPX), 3,7-dimethyl-1-propargylxanthine (DMPX), and 8-(3-chlorostyryl)caffeine (CSC).

In another preferred embodiment, the composition further comprises a chemotherapy drug.

In another aspect the present invention provides a method of treating a patient having a disease or condition associated with expression of ABCG2, comprising administering to the patient a therapeutically effective amount of a composition comprising a pharmaceutically active agent and a xanthine compound, wherein the pharmaceutically active agent is an ABCG2 substrate and the xanthine compound has a structure according to formula (II):

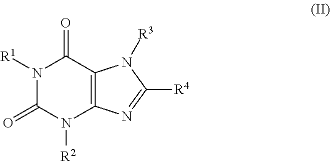

wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen, or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, C₂-C₆ alkenyl, C₂-C₆ alkynyl, aryl, arylalkyl, and arylalkenyl, wherein the alkyl, cycloalkyl, and aryl or aryl part of the arylalkyl and arylalkenyl is optionally substituted by one to five substituents independently selected from the group consisting of halogen, C₁-C₆ alkyl, hydroxyl and C₁-C₆ alkoxy.

In one embodiment of this aspect, the xanthin compound is characterized by Formula II as described above, wherein R¹, R², R³ and R⁴ are not all concurrently hydrogen.

In another embodiment of this aspect, the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, or phenylpropanolamine.

In another embodiment of this aspect, the composition is not coffee, tea, or a caffeinated soft beverage or energy beverage.

In another embodiment of this aspect, the xanthin compound is characterized by Formula II as described above, wherein R¹, R², R³ and R⁴ are not all concurrently hydrogen; the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, phenylpropanolamine; and the composition is not coffee, tea, or a caffeinated soft beverage or energy beverage.

In another embodiment of this aspect, the xanthine compound has a structure of Formula II, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl, preferably, wherein R¹, R², R³ and R⁴ are not all concurrently hydrogen.

In another embodiment of this aspect, the xanthine compound is selected from the group consisting of consisting of theobromine, 8-cyclopentyl-1,3-dipropylxanthine (DPCPX), 3,7-dimethyl-1-propargylxanthine (DMPX), and 8-(3-chlorostyryl)caffeine (CSC).

In another embodiment of this aspect, the disease is a cancer.

In another embodiment of this aspect, the disease is a multi-drug resistant cancer characterized by cancerous cells expressing ABCG2.

In another embodiment of this aspect, the composition is administered orally, intravenously, intraarterially, intramuscularly, intracolonically, intracranially, intrathecally, intraventricularly, intraurethrally, intravaginally, subcutaneously, intraocularly, intranasally, topically, or by any combinations thereof.

In another embodiment of this aspect, the cancer is selected from brain cancer, lung cancer, stomach cancer, duodenal cancer, esophagus cancer, breast cancer, colon and rectal cancer, bladder cancer, kidney cancer, pancreatic cancer, prostate cancer, ovarian cancer, mouth cancer, eye cancer, thyroid cancer, urethral cancer, vaginal cancer, neck cancer, lymphoma, acute lymphocytic leukemia, chronic myelogenous leukemia, chronic lymphocytic leukemia, hairy cell leukemia and myelomas.

In another aspect the present invention provides a method of improving bioavailability of a pharmaceutically active agent delivered across an ABCG2 expressing membrane to a patient in need thereof by administering to the patient the pharmaceutically active agent in combination with a xanthine compound according to formula II:

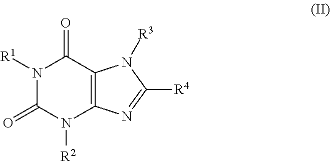

wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen, or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, C₂-C₆ alkenyl, C₂-C₆ alkynyl, aryl, arylalkyl, and arylalkenyl, wherein the alkyl, cycloalkyl, and aryl or aryl part of the arylalkyl and arylalkenyl is optionally substituted by one to five substituents independently selected from the group consisting of halogen, C₁-C₆ alkyl, hydroxyl and C₁-C₆ alkoxy,
wherein the pharmaceutically active agent is an ABCG2 substrate.

In one embodiment of this aspect, the xanthine compound is administered prior to the pharmaceutically active agent.

In another embodiment of this aspect, the xanthine compound is administered simultaneously with the pharmaceutically active agent.

In another embodiment of this aspect, the xanthine compound is administered after the pharmaceutically active agent.

In another embodiment of this aspect, the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, or phenylpropanolamine.

In another embodiment of this aspect, the xanthine compound is not administered in the form of coffee, tea, or a caffeinated soft beverage.

In another embodiment of this aspect, the xanthine compound has a structure characterized by formula II, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl, preferably, wherein R¹, R², R³ and R⁴ are not all concurrently hydrogen.

In another embodiment of this aspect, the xanthine compound is selected from the group consisting of consisting of theobromine, 8-cyclopentyl-1,3-dipropylxanthine (DPCPX), 3,7-dimethyl-1-propargylxanthine (DMPX), and 8-(3-chlorostyryl)caffeine (CSC).

In another embodiment of this aspect, the patient is inflicted with a multi-drug resistant cancer characterized by cancerous cells expressing ABCG2.

In another aspect, the present invention provides use of the composition(s) in any of the embodiments described herein in the manufacture of a medicament for treatment of a disease or condition associated with expression of ABCG2. In a preferred embodiment, the disease is a cancer. In a more preferred embodiment, the disease is a multi-drug resistant cancer characterized by cancerous cells expressing ABCG2.

In other embodiments, the composition containing the xanthine compound may be administered before the chemotherapy drug, e.g., about 72 hours before through one hour before, including without limitations, about 60 hours before, about 48 hours before, about 36 hours before, about 24 hours before, about 16 hours before, about 8 hours before, about 4 hours before, about 3 hours before, about 2 hours before or about one hour before the administration of the chemotherapeutic drug.

In yet other embodiments, the composition containing the xanthine compound may be administered after the chemotherapy drug, e.g., about 72 hours after through one hour after, including without limitations, about 60 hours after, about 48 hours after, about 36 hours after, about 24 hours after, about 16 hours after, about 8 hours after, about 4 hours after, about 3 hours after, about 2 hours after or about one hour after the administration of the chemotherapeutic drug. This embodiment is suitable if the in vivo half lives of the chemotherapeutical agents are long enough and the xanthine compound does not physically bind to the chemo-agents.

The amount of the xanthine compound administered with the single dose of the composition depends on the formulation and the route of administration. For example, as noted above, nanoparticulate formulations provide an increased bioavailability of the active ingredient. Similarly, a localized targeted delivery may result in a need for a lower dose than a systemic administration. In either case, the dose of the xanthine compound should be sufficient to potentiate the effect of the chemotherapeutic drug at the desired location. Thus, in a non-limiting example, assuming a localized tumor and targeted delivery, in one embodiment, the dose of a xanthine compound chosen such that the amount of the xanthine compound at the site of the tumor cells is between about 0.1 and about 15 mM, including 0.2, 0.3, 0.4, 0.5, 0.6, 0.7, 0.8, 0.9, 1, 2, 3, 4, 5, 10, and 15 mM.

The dosages of the chemotherapeutic drugs also depend on the route of administration and the formulation thereof and are well known to practitioners of ordinary skill in the art.

In another aspect, the composition is provided, comprising both the chemotherapeutic drug and the caffeine or the analog thereof. Considering that the disclosure above (e.g., the nature of xanthine compounds, chemotherapeutic drugs, formulations, the dosages and administration routes) is applicable to this composition, no further discussion of this aspect needs to be made.

Essentially any cancer cell line expressing ABCG2 will respond to treatment methods according to the present invention employing the inventive compositions. Cancers susceptible to treatments according to the instant invention include, preferably, solid tumors, such as, for example, brain cancer, lung cancer, stomach cancer, duodenal cancer, esophagus cancer, breast cancer, colon and rectal cancer, bladder cancer, kidney cancer, pancreatic cancer, prostate cancer, ovarian cancer, mouth cancer, eye cancer, thyroid cancer, urethral cancer, vaginal cancer, neck cancer, lymphoma, adenocarcinomas of the digestive tract, endometrium, and lung, melanoma, osteosarcoma, high-grade soft tissue sarcomas, prostate cancer, and the like. In other embodiments, different cancers of blood cells are amenable to treatment. These blood cancers include, without limitations, Acute myeloid leukemia, Acute lymphocytic leukemia, Chronic myelogenous leukemia, Chronic lymphocytic leukemia, Hairy cell leukemia, and myelomas.

In yet another aspect of the invention, a MDR cocktail is provided. The cocktail according to this aspect of the invention is a composition comprising a xanthine compound according to the present invention (as described above) and an inhibitor of at least one protein other than ABCG2 (i.e., the inhibitor of at least one characteristic, such as an amount, an activity, a proper cellular distribution of the protein) that is also responsible for the development of MDR. Such proteins responsible for the development of MDR include, without limitations, P-glycoprotein, multidrug resistance-associated proteins (1-8, 10,11), lung resistance-related protein, ABCA2, ABCB11.

Suitable examples of such inhibitors include, without limitations, Elacidar (GF-120918), Tariquidar (XR-9576), Biricodar (VX-710), XR-9577, and WK-X-34). Additional inhibitors may be found according to assays well known in the art and described below.

Additionally, libraries of compounds may be screened to find out suitable inhibitors. Methods for synthesizing combinatorial libraries and characteristics of such combinatorial libraries are known in the art (See generally, Combinatorial Libraries: Synthesis, Screening and Application Potential (Cortese Ed.) Walter de Gruyter, Inc., 1995; Tietze and Lieb, Curr. Opin. Chem. Biol., 2(3):363-71 (1998); Lam, Anticancer Drug Des., 12(3):145-67 (1997); Blaney and Martin, Curr. Opin. Chem. Biol., 1(1):54-9 (1997); and Schultz and Schultz, Biotechnol. Prog., 12(6):729-43 (1996)).

The cocktail may further comprise a chemotherapy drug (for example, from the list above), which is a substrate to ABCG2 or at least one of the other proteins responsible for the development of MDR. The methods for determining whether the chemotherapy drug of interest is a substrate for ABCG2 or the protein responsible for the development of MDR are known in the an For example, basolateral-to-apical/apical-to-basolateral (B to A/A to B) efflux ratio of the compounds of interest in the cells expressing ABCG2 or another protein responsible for the development of MDR may be used.

In yet another aspect of the invention, xanthine compounds according to the preset invention may be used to increase bioavailability of an orally administered drug. This aspect of the invention stems from the observations that xanthine compounds are effective inhibitors of ABCG2 activity and that ABCG2 is expressed in the apical membrane of the gastrointestinal tract and other membranes across which ABCG2 substrates must be delivered.

A variety of pharmaceutical compositions containing caffeine are known in the art, containing active ingredients such as ergotamine tartrate, acetaminophen, ibuprophen, Isometheptene Mucate, acetylsalicylic acid or a salt thereof, butalbital, propoxyphene, pyrilamine maleate, chlorpheniramine, phenylpropanolamine. It should be noted, however, that in these medications caffeine is included because of its properties as an analgesic or an analgesic adjuvant that may derive from it being a non-selective adenosine antagonist.

Thus, in this aspect of the invention, the instant application provides a composition comprising an orally administered drug which is a substrate for ABCG2 and a xanthine compound according to Formula II, used as an ABCG2 antagonist to increase the bioavailability of the drug that is a substrate for ABCG2. Also provided is a use of a xanthine compound according to Formula II for a manufacture of a medicament for increased bioavailability of an orally administered drug which is a substrate for ABCG2.

The orally administered drugs are well known and include, without limitation, drugs which are ABCG2 substrates within the following categories of drugs: analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, calcium channel blockers, contrast media, corticosteroids, cough suppressants, diagnostic agents, diagnostic imaging agents, diuretics, dopaminergics, endogenerous substances, haemostatics, immuriological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, vasodilators, and any other agents that are substrates of ABCG2.

In addition to the examples of suitable chemotherapeutic drugs, non-limiting examples of suitable compounds include Zidovudine (AZT), Lamivudine, Abacavir, Acyclovir, Atorvastatin, Pravastatin, Rosuvastain, Pitavastatin, Cerivastatin, Genistein, Quercetin, Benzo[a]pyrene-3-sulfate Benzo[a]pyrene-3-glucuronide, Estrone-3-sulfate, 4-Methylumbelliferone sulfate, 4-Methylumbelliferone, 6-Hydroxy-5,7-dimethyl-2-methylamino-4-(3-pyridylmethyl)benzothiazole glucuronide (E3040) glucuronide, Dehydroepaindrosterone sulfate, 17-β-estradiol sulfate, 17-β-estradiol glucronide, Acetaminophen sulfate, Troglitazone sulfate, Afluzosin, Albendazole sulfoxide, Oxfendazole, Pantoprazole, Ciprofloxacin, Danofloxacin, Diclofenac, Glyburide, Leflunomide, Ofloxacin, Norfloxacin, Sulfasalazine, Teriflunomide, Erythromycin, Dirithromycin, Rifampicin, Nitrofurantoin, Enrofloxacin, Gepafloxacin, Ulifloxacin, Dihydropyridine, Dihydrotestosterone, Sulfasalazine, Phenethyl isothiocyanate, Azidopine, Nitrendipine, Dipyridamole, Ochra-toxin A, GV-196771, Folic acid, Vitamin K3, Protoporphyrin IX, Uric acid, Cimetidine, Riboflavin, ME-3229, JNJ-7706621 and any combinations thereof.

The composition of this aspect of the invention may be prepared based on the disclosure above, since the discussion of formulations, dosages, timing of administration, and nature of the caffeine analogs are also applicable hereto.

The methods of determining whether a given substance (e.g., the chemotherapeutic drug) is a substrate for ABCG2 are known in the art. For example, in one embodiment, efflux activity of ABCG2 may be evaluated by monitoring the basolateral-to-apical/apical-to-basolateral (B to A/A to B) efflux ratio of the compounds of interest in a cell line expressing ABCG2.

The present invention therefore also includes the use of a xanthine compound in the preparation of a medicament containing a drug that is a substrate for ABCG2 to improve the bioavailability of the drug (and/or reverse or prevent ABCG2 mediated multi-drug resistance), wherein the property of the drug being a substrate for ABCG2 is determined by measuring the basolateral-to-apical/apical-to-basolateral efflux ratio of the drug in a cell line expressing ABCG2.

Moreover, the xanthine compounds of the present invention can also be used, in some embodiments maybe preferably, in conjunction with another ABCG2 inhibitor or inhibitors. Suitable ABCG2 inhibitors include, without limitations, Abacavir, AG1478, Amprenavir, Atazanavir, Biricodar (VX-710), Cannabinol (CBN), Cannabidiol (CBD), Ciclosporine A, Chrysin, Curcumin 1, Delavirdine, Dipyridamole, Dofequidar fumarate, Efavirenz, Ko132, Ko134, Ko143, Lopinavir, Nicardipine, Nelfinavir, Novobiocin, Omeprazole, Pantoprazol, Phenylchrysin, Querceptin, Ritonavir, Sirolimus, Saquinavir, Tectochrysin, Tacrolimus, Delta 9-tetrahydrocannabinol, Tetrahydrocurumin, PZ-39, Erlotinib, GF120918 (elacridar), Fumitremorgin C (FTC), Gefitinib, Imatinib, butorylamides and synthetic analogs of butorylamide F, dimethoxyaurones, non-basic chalcone analogues, acridones, ginsenosid metabolites, piperazinobenzopyranones, and phenalkylaminobenzopyranones, several synthesized dihydropyridines, flavonoids (e.g., silymarin, hesperetin, quercetin, and daidzein), and the stilbene resveratrol (H. E. M. zu Schwabedissen and H. K. Kroemer in Drug Thansporters (2011)).

Even though it is known that caffeine activates multiple signal transduction pathways, without wishing to be bound by theory, the inventors propose that the effect of caffeine or analogs thereof is mediated by Phosphoinositide 3-kinase (PI3K) pathway. This pathway is known to involve AKT kinase and mTor with implications of involvement in cancer. Therefore, it is feasible that inhibitors of PI3K and compounds downstream of PI3K may also be useful for all aspects of this invention.

The suitable non-limiting examples of inhibitors of PI3K include edelfosine (ET-18-OCH3), LY294002, LY303511, Quercetin Dihydrate, and Wortmannin.

The inhibitors of Akt include, without limitations, Akt inhibitors GSK2110183 and SR13668 (two orally bioavailable akt inhibitors listed on the NCI Drug Dictionary), SH-5 (Akt inhibitor 11, CALBIOCHEM Inc., LA JOLLA, Calif.), SH-6 (Akt inhibitor III, CALBIOCHEM Inc.), API-2 (Akt inhibitor V, CALBIOCHEM Inc.), FPA124, KP372-1, Akt inhibitor IV, NL-71-101, and the like. Other Akt inhibitors can be found in CALBIOCHEM Inc. source documents, which are incorporated by reference herein.

### Definitions

The term “about,” as used herein, refers to a range of values within ten percent (10%) of a baseline value. Thus, for example, the phrase “about 100” refers to a range of values between 90 and 110.

The term “bioavailability,” as used herein, refers to the amount of a drug at a site within the patient, where the effect of the drug is desired, and includes, without limitations, the amount of a drug within a cell, e.g., cancer cell. The term “bioavailability” also refers to the fraction of the total amount of the drug in the bloodstream.

The term “alkenyl,” as used herein, refers to a group derived from a straight or branched hydrocarbon chain having one or two C═C double bonds therein. Representative examples of C₂-C₆ alkenyl group include, but are not limited to, vinyl, allyl, 1-propenyl, 1-buten-4-yl, and 2-penten-1-yl.

The term “alkoxy,” as used herein, refers to an “RO—” group, where “R” is an alkyl, preferably C₁-C₆ alkyl. Representative examples of alkoxy group include, but are not limited to, methoxy (CH₃O—), ethoxy (CH₃CH₂O—), and t-butoxy ((CH₃)₃CO—).

The term “alkyl,” as used herein, refers to a group derived from a straight or branched saturated hydrocarbon chain. Representative examples of C₁-C₆ alkyl group include, but are not limited to, methyl, ethyl, isopropyl, and tert-butyl.

The term “alkynyl,” as used herein, refers to a group derived from a straight or branched chain hydrocarbon comprising at least one carbon-carbon triple bond (—C≡C—). Representative examples of C₂-C₆ alkynyl group include, but are not limited to, acetylenyl (HC≡C—), 1-propynyl (CH₃C≡C—), and propargyl (HC≡CCH₂—).

The term “aryl,” as used herein, refers to a phenyl or naphthyl group, preferably phenyl group, optionally substituted by one to five substituents independently selected from C₁-C₆ alkyl, C₁-C₆ alkoxy, hydroxyl, and halogen.

The term “arylalkyl,” as used herein, refers to an alkyl group substituted with an aryl group, wherein aryl part of the arylalkyl group may optionally be substituted by one to five substituents independently selected from, but not limited to, C₁-C₆ alkyl, C₁-C₆ alkoxy, hydroxyl, and halogen. Represented examples of arylalkyl include, but are not limited to, benzyl and 2-phenyl-1-ethyl (PhCH₂CH₂—).

The term “arylalkenyl,” as used herein, refers to a C₂-C₆ alkenyl group substituted by an aryl group, wherein aryl part of the arylalkenyl group may optionally be substituted by one to five substituents independently selected from, but not limited to, C₁-C₆ alkyl, C₁-C₆ alkoxy, hydroxyl, and halogen. Representative examples of arylalkenyl include, but are not limited to, styryl (PhCH═CH₂—) and phenylallyl (PhCH═CHCH₂—).

The term “cycloalkyl,” as used herein, refers to a group derived from a saturated carbocycle, by removal of a hydrogen atom from the saturated carbocycle. Representative examples of cycloalkyl groups include, but are not limited to, cyclopropyl, cyclopentyl, and cyclohexyl.

The term “halogen,” as used herein, refers to F, Cl, Br, or I.

The terms “hydroxy” or “hydroxyl,” as used herein, refer to —OH.

The terms “treat,” “treatment” and the like refer to executing a protocol, which may include administering one or more drugs to a patient (human or otherwise), in an effort to alleviate signs or symptoms of the disease. Alleviation can occur prior to signs or symptoms of the disease appearing, as well as after their appearance. In addition, “treating” or “treatment” does not require complete alleviation of signs or symptoms, does not require a cure, and specifically includes protocols which have only a marginal effect on the patient.

In this instance, treatment involves use of this invention as a single delivery therapeutic, or multiple or repeated delivery therapeutic, or a control delivery therapeutic and is meant to be delivered locally, systemically, intravascularly, intramuscularly, intra-peritoneally, inside the blood-brain barrier, or via other various routes.

For example, the term “cancer treatment” may refer on a cellular level to a reduced rate of tumor growth and/or increased apoptosis of tumor cells, compared to untreated cells or cells treated with vehicle. According to this definition, the growth is reduced by at least 10% (e.g., 25%, 50%, 75%, 80%, 85%, 90%, 95%, or 99%) or the apoptosis is increased by at least 10% (e.g., 25%, 50%, 75%, 100%, 150%, 200%, etc).

The term “patient” refers to a biological system to which a treatment can be administered. A biological system can include, for example, an organ, a tissue, or a multi-cellular organism. A patient can refer to a human patient or a non-human patient.

The xanthine compound may be present in a composition in different formulations including modified release formulations and/or nanoparticulate formulations. Examples of such formulations have been described in the art. In this application, the term “xanthine compound” and “caffeine or caffeine analog” are often used interchangeably, in either case without any intention to be limited whatsoever.

The advantages of the nanoparticulate formulation include an increased rate of dissolution in vitro, an increased rate of absorption in vivo, a decreased fed/fasted ratio variability, and a decreased variability in absorption.

The main advantage of the modified release formulations is that the drug or drugs are released according to the pre-determined profile, thus eliminating the necessity of multiple administrations.

Suitable pharmaceutically acceptable carriers are well known to those skilled in the art. These include non-toxic physiologically acceptable carriers, adjuvants or vehicles for parenteral injection, for oral administration in solid or liquid form, for rectal administration, nasal administration, intramuscular administration, subcutaneous administration, and the like.

The composition of the instant invention may be used for the preparation of a medicament adapted for administration via different routes. A practitioner of the invention (e.g., a physician) would be able to select the most appropriate route of administration considering the individual needs of the patient and the location of the cancer. Without limitations, the envisioned administration routes include oral, intravenous, intra-arterial, intramuscular, intracolonic, intracranial, intrathecal, intraventricular, intraurethral, intravaginal, sub-cutaneous, intraocular, topical, intranasal, and any combinations thereof.

The composition of the instant invention may be administered simultaneously (i.e., within one hour, or within 30 minutes, or within 15 minutes, or within 10 minutes or within 5 minutes or one minute or at the same time with the chemotherapeutic drug of choice, such as, for example, anthracyclines, campothecins, indolocarbazones, antifolates, tyrosine kinase inhibitors, and other agents.

Specific drugs which are substrates for ABCG2 include, without limitations, chemotherapy drugs such as Mitoxantrone, BBR3390, Daunorubicin, Doxorubicin, Epirubicin, Bisantrene, Flavopiridol, Etoposide, Teniposide, 9-Aminocamptothecin, Topotecan, Irinotecan, SN-38, SN-38 glucuronide, Diflomotecan, Homocamptothecin, karenitecin (BNP 1350), gimatecan, exatecan (DX-891f), DX-8951f, BNP-1350, ST-1976, ST-1968, J-107088, NB-506, Compound A, UNC-01, Methotrexate, methotrexate, di-and triglutamate, GW-1843, Tomudex, Imatinib, Gefitinib, CI-1033, Nilotinib, desatinib, sunitinib, erlotinib, Triazoloacridones, and any combinations thereof.

The invention will now be illustrated in the following non-limiting examples.

### EXAMPLES

### Western Blotting

Cells were washed twice with cold phosphate-buffered saline and lysed in RIPA lysis buffer plus protease inhibitor. Protein concentrations of the cell lysates were determined using the bicinchoninic acid (BCA) protein assay as manufacturer's description. Equal amounts of total protein (5 to 15 μg) were analyzed by 8% sodium dodecyl sulfate-polyacrylamide gel electrophoresis followed by immunoblotting using mouse monoclonal antibody (clone BXP-21) against ABCG2 (1:1,000; Kamiya), rabbit monoclonal antibody against GAPDH (1:1000; Cell Signaling). The secondary antibody was either horseradish peroxidase-conjugated goat-anti-mouse IgG (1:2,500; Amersham) or horseradish peroxidase-conjugated goat anti-rabbit immunoglobulin G (IgG; Santa Cruz Biotechnology). Immunoreactive bands were visualized using an enhanced chemiluminescent system (Pierce) according to the manufacturer's recommendations.

### Immunochemical Staining

Cells grown on glass coverslips were washed three times with PBS, fixed in 4% paraformaldehyde solution and permeablized in 0.2% Triton-X-100 solution each for 10 min. Cells were washed with PBS three times at each interval. Cells were then incubated with 2% BSA in 0.1% Triton X-100 PBS buffer at room temperature for 1 h and followed by incubation with monoclonal ABCG2 antibodies (BXP-21; diluted 1:250; Kamiya) containing 0.1% Triton X-100 in a humid chamber. After being washed three times with PBS, the cells were incubated with Alexa Fluor® 488-conjugated goat anti-mouse IgG at 37° C. for 1 h. The cells were then mounted and sealed with DAPI mounting medium onto glass slides and observed under a Zeiss confocal microscope (Ina, Japan).

### Efflux Assay

Cells were collected and suspended in phenol red-free complete medium alone, or complete medium containing 500 nM Bodipy-prazosin with or without 10 μM FTC and incubated at 37° C. in 5% CO₂ for 30 min. The incubations were stopped immediately by adding 4 ml cold PBS to the cell suspension. The cells were then washed three times with ice-cold PBS and incubated for 1 h at 37° C. in 5% CO₂ in complete media with or without 10 μM FTC. After the incubation, cells were then washed with cold PBS for 3 times and subjected to the Coulter Cytomics FC500 Flow Cytometer with a 488-nm argon laser and 530-nm band pass filter to analyze the individual intracellular fluorescence intensity.

### Apoptosis Assay

The Guava EasyCyte flow cytometry analysis (Guava Technologies, Hayward, Calif.) was utilized to analyze the apoptotic cells. The assays were conducted according to the manufacture's instruction. Briefly, total cells were collected and washed with cold PBS. Then 5 μL of annexin V-phycoerythrin, a marker for early apoptosis, and 5 μL of 7-amino-actinomycin (7-AAD), a cell-impermeant dye indicating late apoptosis or dead cells (Guava PCA-96 Nexin Kit) were added to the cell suspensions. After 20 mins incubation and thorough mixing, the samples were analyzed on a Guava PC and data were collected.

### Animal Experiment

Both drug resistant and drug sensitive xenografts (50-150 mm³ in volume) were established in the same female nude mice (BalbC, nu/nu) by subcutaneous implantation of cells of MCF7/mx100 in one flank and MCF7/wt in the other. Animals were monitored daily and tumor volume was estimated by caliper measurements: [tumor volume=(length×width²)/2]. Once the xenografts were established, mice were grouped into 3 cohorts of 5 mice. Each of 2 cohorts received caffeine at 50 mg/kg, 100 mg/kg, respectively, while the control cohort received carrier alone. Caffeine was administered by i.p. and 18-20 hours after initial caffeine treatment (day 0), all mice were administered 1.0 mg/kg mitoxantrone by i.v. This was repeated twice weekly. Animal weight and tumor volume was recorded every 7 days after the initiation of mitoxantrone administration. The drug sensitive xenografts served as an internal positive control for mitoxantrone action, while the drug resistant xenografts were examined for combined therapeutic effects of caffeine and mitoxantrone by comparing experimental cohorts with the control cohort. Results were expressed as a percentage of the tumor volume at the day of measurement over the volume at day 0. At the end of study, animals were sacrificed and tumors were excised and analyzed for ABCG2 expression.

The same cohorts were repeated for the adenosine receptor antagonists DPCPX and DMPX, after the effective but yet nontoxic concentrations were determined by the preliminary study for these two compounds.

### Example 1

### Caffeine Down-Regulates Protein Level of Gene ABCG2 in Placenta In Vitro and the Effect is Reversible

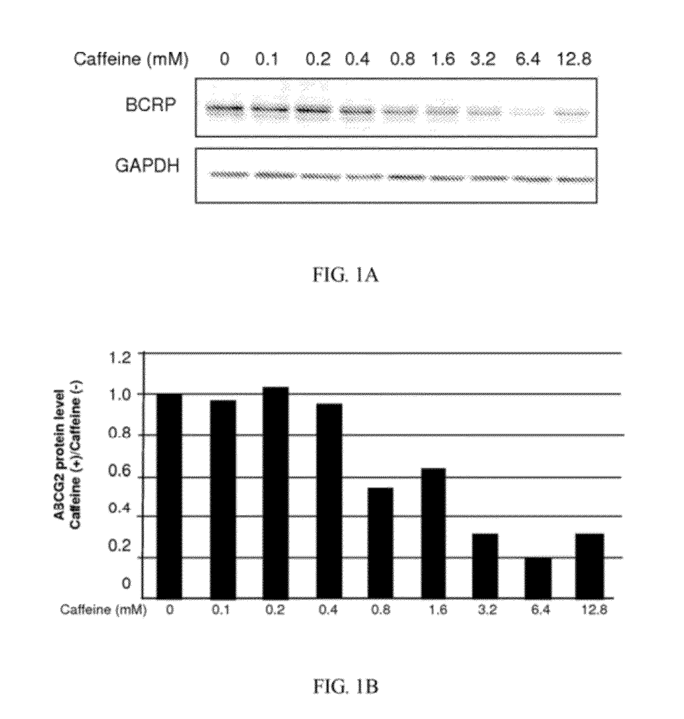

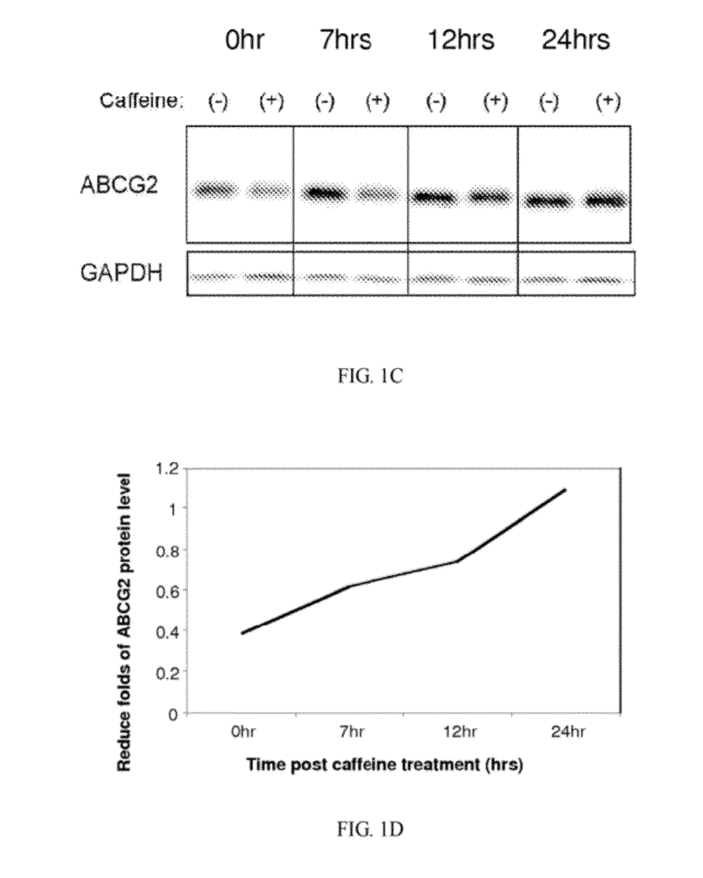

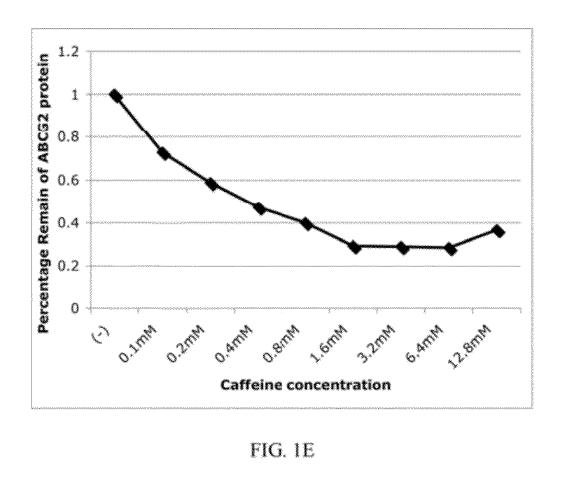

In experiments to test the effect of caffeine on ABCG2 gene expression, the placental cell line Bewo maintained in F-12K medium (ATCC, #30-2004) supplemented with 10% heat-inactivated fetal bovine serum (Atlanta Biologicals, GA) at 37° C. in a 5% (v/v) CO₂ atmosphere was treated at 60-70% confluency with caffeine at increasing concentrations from 0.1 mM to 14 mM for 24 hrs. After treatment, the ABCG2 protein was analyzed by western-blotting using GAPDH as a protein loading control. The ABCG2 protein begins to decrease when the caffeine concentration is at 0.8 mM and caffeine continues to reduce this protein in a dose dependent manner, as illustrated in FIGS. 1A-1E. FIG. 1A shows the dose response profile. Cells were treated with caffeine at eight concentration levels indicated in the figure. The whole cell lysate was prepared after treatment and subjected to the western blotting to analyze protein level of ABCG2 using a monoclonal antibody BXP-21 (Kamiya Biomedical company). The 72 kD band was identified to be ABCG2 and GAPDH was used as loading control. FIG. 1B is a bar graph of the quantification of the western blotting shown in FIG. 1A, protein level of ABCG2 is normalized with GAPDH protein level.

FIGS. 1C and 1D demonstrate the reversibility of caffeine effect. After 24 hrs of caffeine (14 mM) treatment, fresh medium was added and cells were cultured for another 24 hrs. The level of ABCG2 protein was determined 7, 12, and 24 hours after the fresh medium was added. After 24 hours, the amount of ABCG2 protein returned to the level of ABCG2 protein in non-treated cells, as illustrated in FIG. 1D.

### Example 2

### Caffeine Treatment Altered Subcellular Localization of ABCG2 Protein

To verify the western blotting data and to further investigate caffeine regulation of ABCG2 protein, an immunofluorescence staining was carried out. The Bewo cells cultured as in Example 1 were treated with caffeine either at four different concentrations or at 7 mM for different time periods as indicated and then probed with BXP-21 monoclonal antibody. In non-treated cells, ABCG2 was located on the cell membrane and an aggregation spot of ABCG2 protein near the nucleus was observed, consistent with observations from previous studies.

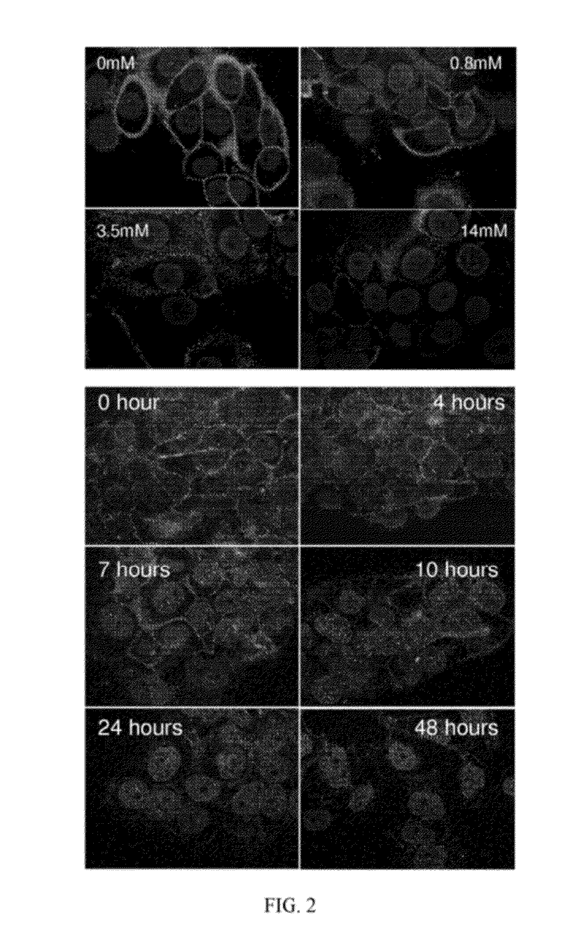

When treated with caffeine, besides the decrease in total amount of protein, the membrane localized form of ABCG2 decreased significantly and the rest of the protein diffused into cytoplasm, the peak time of which is at 10 hours of caffeine treatment (FIG. 2). However, with the technique used, it is unclear which subcellular compartment the ABCG2 protein aggregation belongs to in the untreated cells and where ABCG2 diffused into after caffeine treatment.

### Example 3

### Caffeine does not Alter ABCG2 mRNA Level

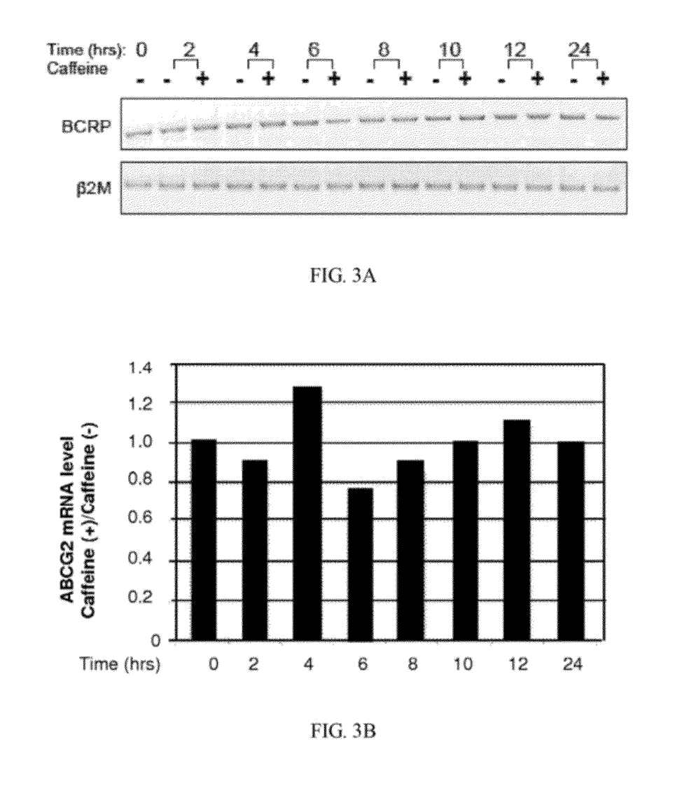

Bewo cells were cultured as in Example 1. Cells were treated with caffeine for times indicated prior to RNA preparation and RT-PCR was performed to analyze mRNA of ABCG2 using primers hBCRP1For/hBCRP1-Rev (hBCRP1-For: CCATAGCAGCAGGTCAGAGT (SEQ ID NO: 1): hBCRP1-Rev: AGGCCACGTGATTCTTCCAC (SEQ ID NO: 2)). Caffeine (14 mM) has no significant effect on ABCG2 mRNA level, as illustrated in FIG. 3.

### Example 4

### Caffeine Increased Intracellular Retention of an ABCG2-Specific Substrate

The inventors investigated the cellular accumulation of a specific ABCG2 substrate when cells were treated with or without caffeine using flow cytometry. MCF-7/MX100 and its parental cells MCF-7 were treated with 14 mM caffeine for 24 hours, then collected the cells and incubated with the ABCG2 specific fluorescence substrate Bodipy-prazosin. The efflux of Bodipy-prazosin was then allowed in the fresh medium incubation, where the intracellular concentration of the Bodipy-parzosin decreases depending on the number and activity of ABCG2 transporter on the plasma membrane.

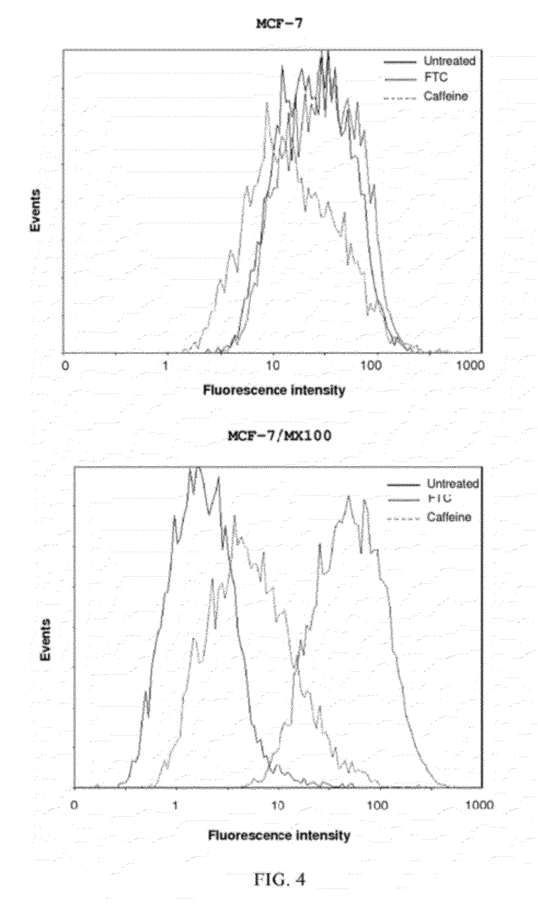

FIG. 4 shows that MCF-7/MX100 cells had significantly increased accumulation of Bodipy-prazosin under caffeine treatment whereas the untreated cells exhibited only basal level of substrate accumulation. On the other hand, in the MCF-7 parental cell line, which has low-to-undetectable ABCG2 expression, both caffeine untreated and treated cells had similar intracellular fluorescence intensity, suggesting that the increased substrate accumulation by caffeine is mediated by downregualtion of ABCG2. Fumitremorgin C (FTC) was used as the positive control, since it has been shown that FTC completely inhibits ABCG2 activity at 10 μM.

### Example 5

### Caffeine Sensitized the ABCG2-Expressing Cells to Mitoxantrone

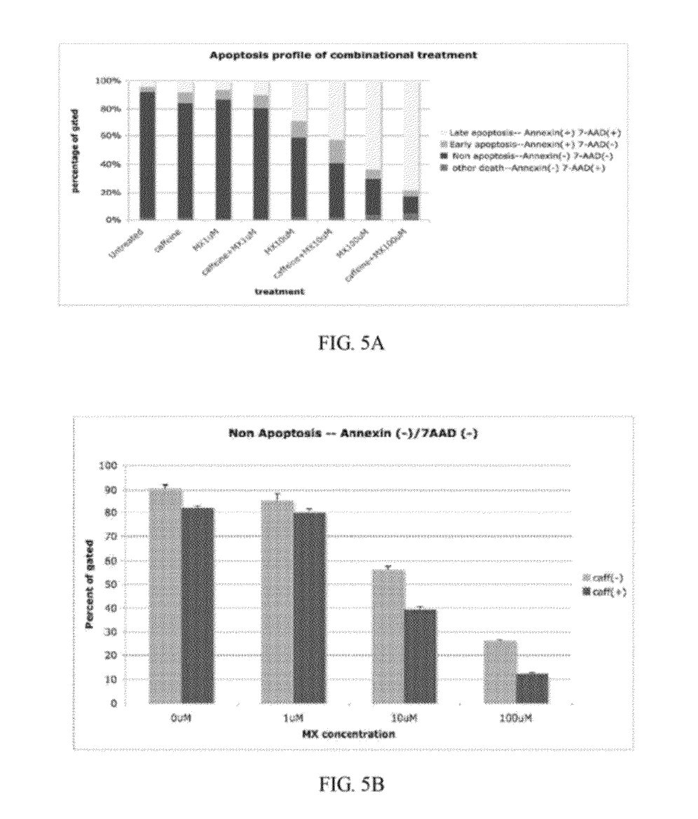

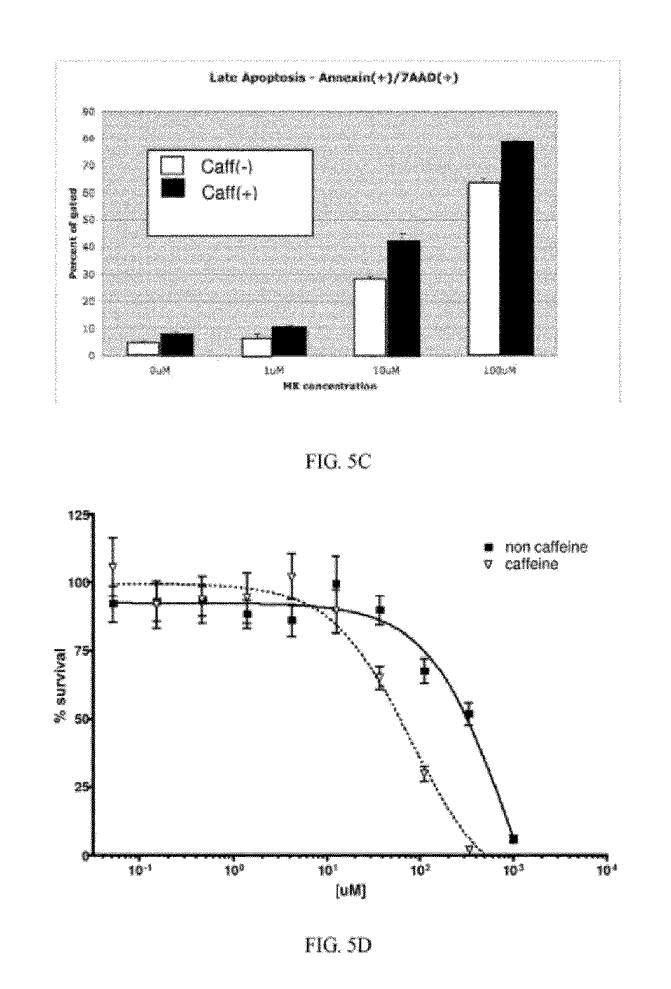

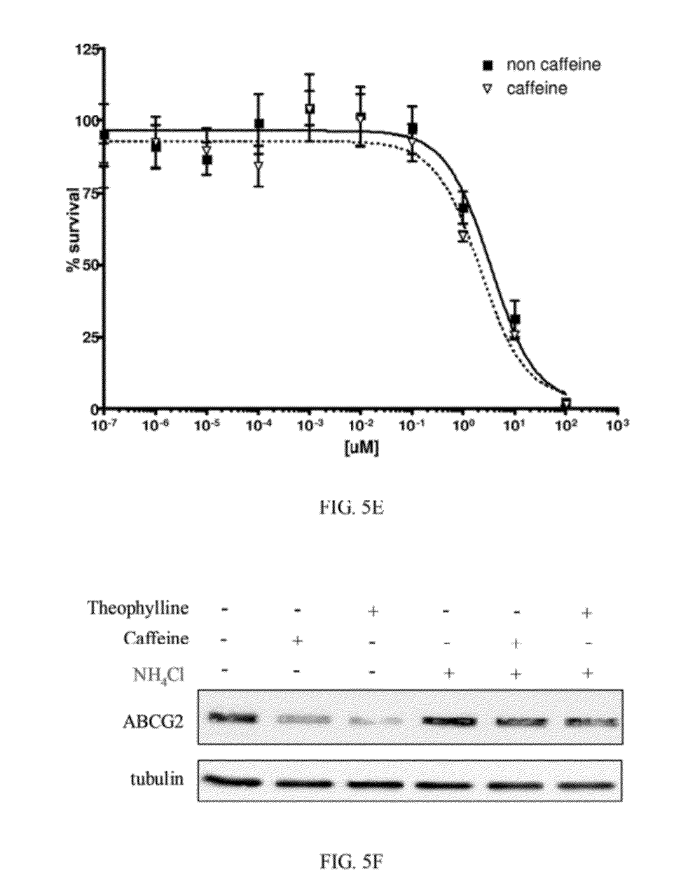

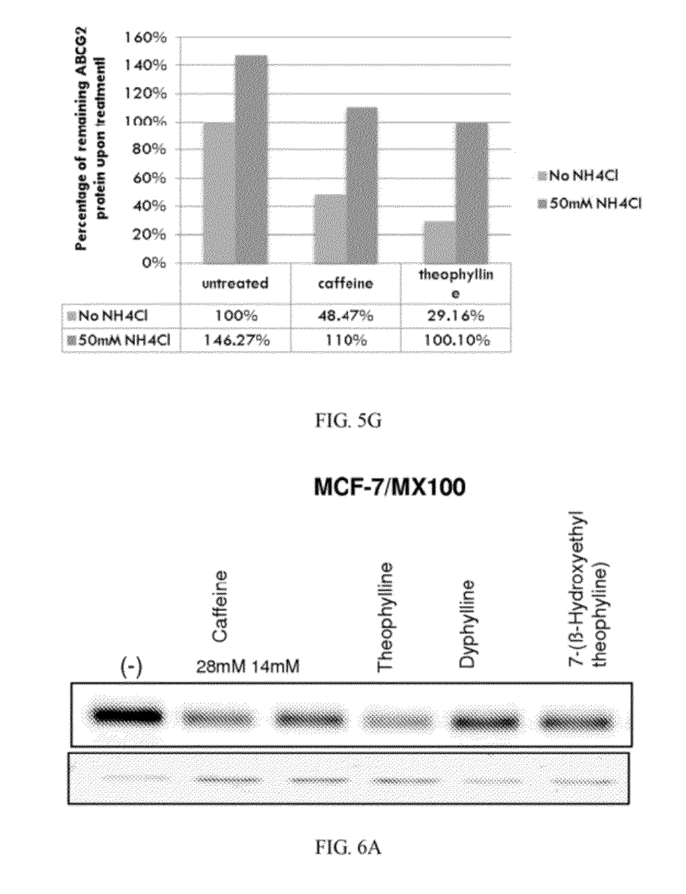

Bewo cells were treated with increasing concentrations of mitoxantrone for 24 hrs, following a 24 hrs treatment of 14 mM caffeine. As discussed above, this concentration was sufficient to decrease the level of ABCG2 protein. All the cells were subjected to analysis for apoptosis profile by Guava Nexin assay. Results are shown in FIG. 5.

The caffeine treated cells had higher percentage of apoptosis and lower living cell percentage than the untreated ones. FIG. 5A shows a bar graph of the apoptosis profile, at 10 μM and 100 μM mitoxantrone treatment, caffeine increases the apoptotic population by 18%˜20%, whereas at 0 μM mitoxantrone, caffeine only caused a minor increase in the apoptosis.

FIG. 5B shows a graph on the healthy, non-apoptosis population, and FIG. 5C shows a graph on the late-apoptosis population.

In addition, the effects of caffeine on the IC50 of mitoxantrone were compared between a non ABCG2 expressing cell line MCF-7 and a drug resistance subline MCF-7/MX100, which highly express ABCG2. As shown in FIG. 5D, caffeine sensitized the MCF-7/MX100 cells to mitoxantrone by decreasing its IC50 by more than 10 fold. However, in MCF-7/sensitive cells, the IC50 of mitoxantrone was not changed significantly by caffeine (FIG. 5E).

Mechanistic study indicated that xanthines accelerates lysosomal degradation of ABCG2 (see FIG. 5F and FIG. 5G). Similar results have been obtained with leupeptin and Bafilomycin.

### Example 6

### Effects of Caffeine Analogs on the ABCG2 Gene Expression

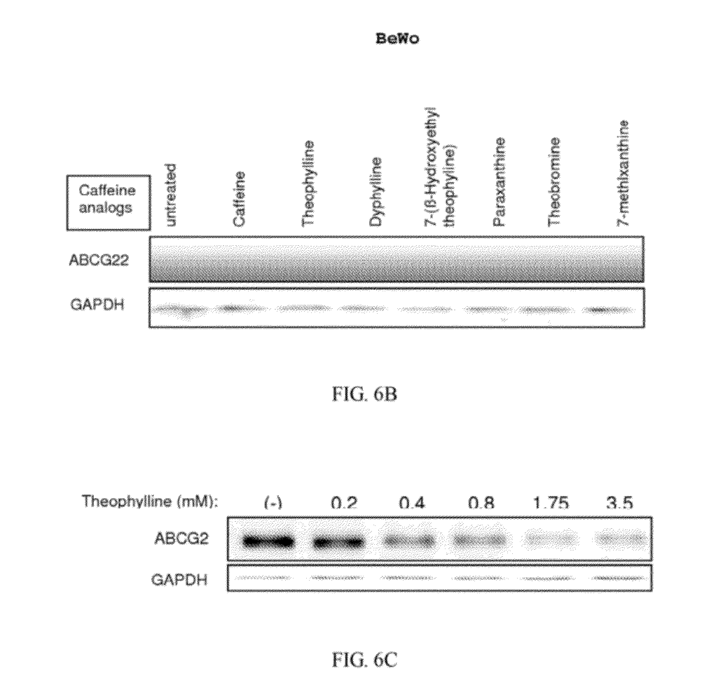

Bewo cells were cultured as described in Example 1. Caffeine analogs theophylline, pentoxifylline, iso-caffeine, Dyphylline, 7-(β-Hydroxyethyl)theophylline, Theobromine, and 7-methlxanthine were utilized to treat the Bewo cells, and the ABCG2 protein level after treatment were examined by western blotting. The results of these experiments are illustrated in FIGS. 6A-6C. Theophylline is the most potent analog among the ones tested.

### Example 7

### PI3K Inhibitor LY294002 Decreases the Concentration of ABCG2 Protein

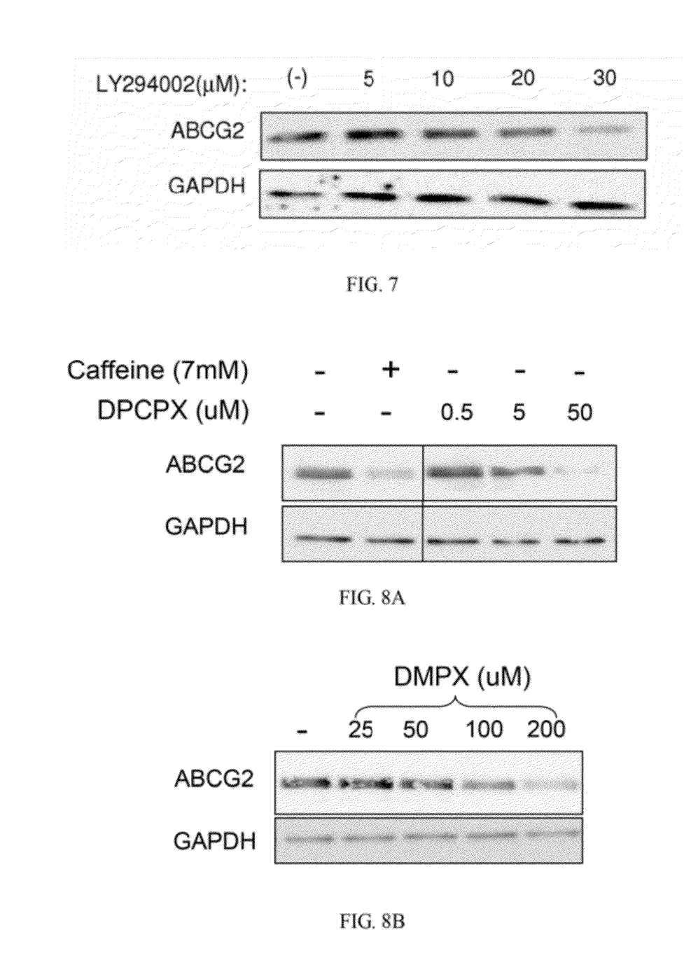

The cells cultured as described in Example 1 were treated with increasing concentrations of the PI3K inhibitor LY294002 for 24 hours and then collected and analyzed by western blot. As shown in FIG. 7, LY294002 downregulated ABCG2 expression in a dose-dependent manner under non-cellular toxic concentrations. GAPDH was used as loading control.

### Example 8

### Effects of Caffeine Analogs DPCPX and DMPX on the Level of ABCG2 Protein

The cells cultured as described in Example 1 were treated with increasing concentrations of DPCPX and DMPX, respectively, for 24 hours and then collected and analyzed by western blot. As shown in FIG. 8A and FIG. 8B, DPCPX and DMPX decreased the level of ABCG2 protein. GAPDH was used as loading control.

### Example 9

### Caffeine and Caffeine Analogs, as Adenosine Receptor Antagonists, Decrease the Level of ABCG2 Protein

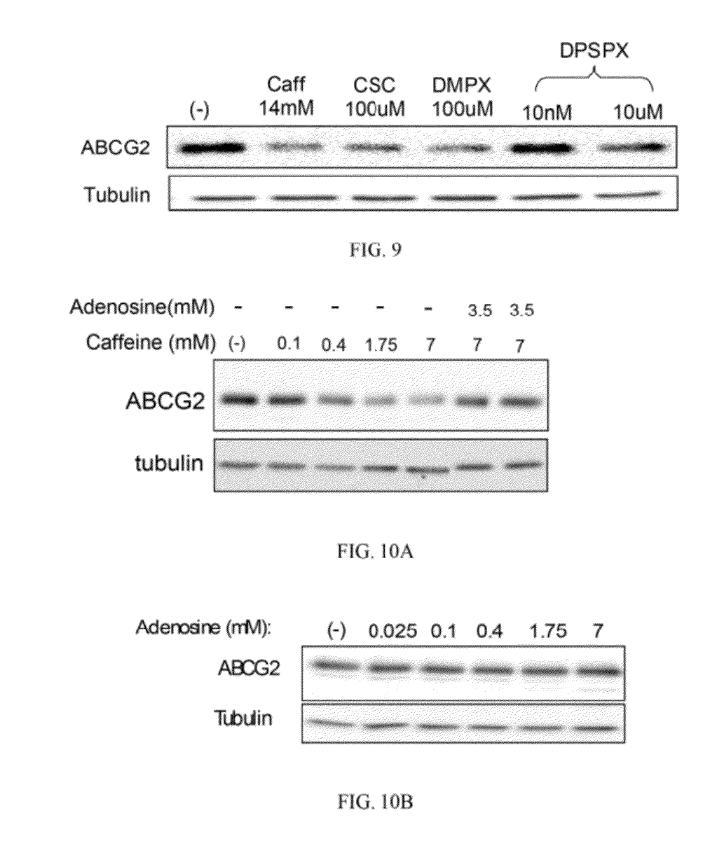

The cells cultured as described in Example 1 were treated with caffeine or caffeine analogs CSC, DMPX, and DMPX, respectively, for 24 hours and then collected and analyzed by western blot. As shown in FIG. 9, these AR antagonists decreased the level of ABCG2 protein. Tubulin was used as loading control.

### Example 10

### Adenosine Reverses Caffeine-Mediated Downregulation of ABCG2

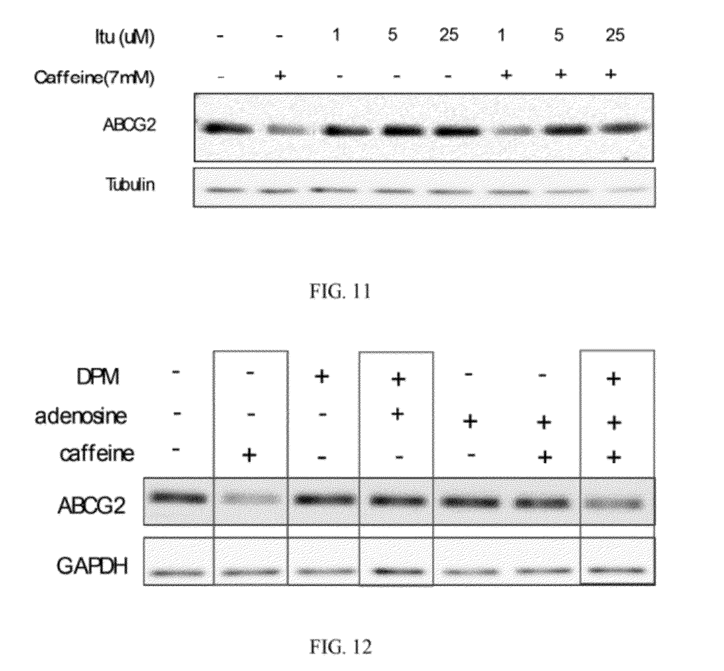

The cells cultured as described in Example 1 were treated with increasing concentrations (0, 0.1, 0.4, 1.75 and 7 mM) of caffeine for 24 hours and then collected and analyzed by western blot. As shown in FIGS. 10A and 10B, treatment with increasing concentration of caffeine gave a decreasing level of ABCG2 protein. However treatment of the 7 mM caffeine-mediated cells with adenosine reversed the downregulation of ABCG2, as shown in FIGS. 10A-10B and FIG. 11. Adenosine phosphorylation is required for xanthines to downregulate ABCG2. Tubulin was used as loading control.

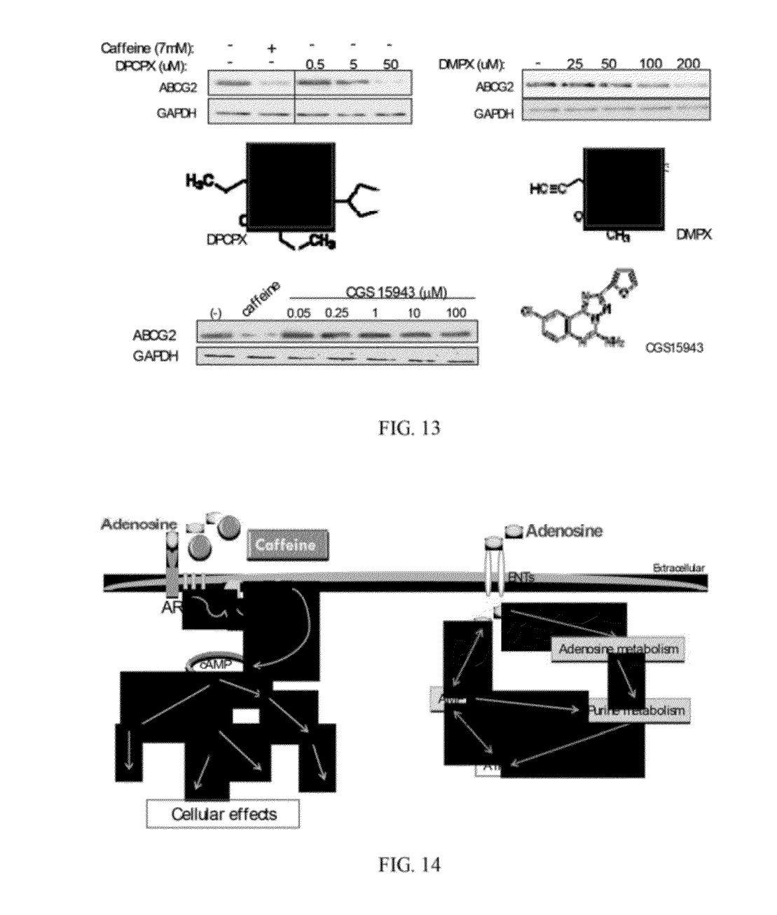

Other experiments have shown that nucleoside transporter inhibition prevented adenosine from reversing the effect of caffeine (see FIG. 12), which indicates that adenosine must be transported into the cell to prevent caffeine from downregulating ABCG2. Moreover, it was found that only adenosine receptor antagonists that are xanthines decrease ABCG2 protein (see FIG. 13).

Various mechanisms of action have been proposed for xanthines. The present inventors hypothesize that xanthines interfere with adenosine metabolism and AMP generation, triggering downstream signaling pathways that in turn induce lysosomal degradation of ABCG2. The adenosine mediated signaling pathways are illustrated in FIG. 14.

### INDUSTRIAL APPLICABILITY

The invention has applications in connection with treating or preventing multi-drug resistance in patients, such as cancer patients, and also with improving bioavailability of drugs.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

All patent and non-patent publications cited in this specification are indicative of the level of skill of those skilled in the art to which this invention pertains. All these publications and patent applications are herein incorporated by reference to the same extent as if each individual publication or patent application was specifically and individually indicated to be incorporated herein by reference.

What is claimed is:

1. A pharmaceutical composition comprising a pharmaceutically active agent and a xanthine compound, wherein the pharmaceutically active agent is an ABCG2 substrate and the xanthine compound has a structure according to formula (II): 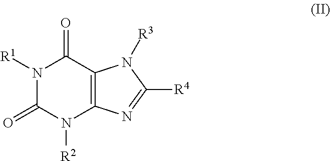
wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, C₂-C₆ alkenyl, alkynyl, aryl, arylalkyl, and arylalkenyl, wherein the alkyl, cycloalkyl, and aryl or aryl part of the arylalkyl and arylalkenyl is optionally substituted by one to five substituents independently selected from the group consisting of halogen, C₁-C₆ alkyl, hydroxyl and C₁-C₆ alkoxy,
with the provisos that the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, or phenylpropanolamine, when the xanthine compound is caffeine, and
that the xanthine compound is not a component of coffee, tea, or a caffeinated soft beverage.

2. The composition of claim 1, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl.

3. The composition of claim 1, wherein the xanthine compound is selected from the group consisting of theobromine, 8-cyclopentyl-1,3-dipropylxanthine (DPCPX), 3,7-dimethyl-1-propargylxanthine (DMPX), and 8-(3-chlorostyryl)caffeine (CSC).

4. The composition of claim 1, wherein the pharmaceutically active agent is selected from the group consisting of analgesics, anti-inflammatory agents, anthelmintics, anti-arrhythmic agents, antibiotics, anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives, astringents, beta-adrenoceptor blocking agents, calcium channel blockers, contrast media, corticosteroids, cough suppressants, diagnostic agents, diagnostic imaging agents, diuretics, dopaminergics, endogenous substances, haemostatics, immuriological agents, lipid regulating agents, muscle relaxants, parasympathomimetics, parathyroid calcitonin, prostaglandins, radio-pharmaceuticals, sex hormones, anti-allergic agents, stimulants, sympathomimetics, thyroid agents, and vasodilators.

5. The composition of claim 1, wherein the pharmaceutically active agent is a chemotherapy drug.

6. The composition of claim 5, wherein the chemotherapy drug is selected from the group consisting of topoisomerase I inhibitors, topoisomerase II inhibitors, camptothecins, mitoxantrone, bisantrene, anthracyclines, indolocarbazones, antifolates, and tyrosine kinase inhibitors.

7. The composition of claim 5, wherein the chemotherapy drug is selected from the group consisting of Mitoxantrone, BBR3390, Daunorubicin, Doxorubicin, Epirubicin, Bisantrene, Flavopiridol, Etoposide, Teniposide, 9-Aminocamptothecin, Topotecan, Irinotecan, SN-38, SN-38 glucuronide, Diflomotecan, Homocamptothecin, karenitecin (BNP 1350), gimatecan, exatecan (DX-8910, DX-8951f, BNP-1350, ST-1976, ST-1968, J-107088, NB-506, Compound A, UNC-01, Methotrexate, methotrexate, di-and triglutamate, GW-1843, Tomudex, Imatinib, Gefitinib, CI-1033, Nilotinib, desatinib, sunitinib, erlotinib, Triazoloacridones, and any combinations thereof.

8. The composition of claim 1, wherein the pharmaceutically active agent is an inhibitor of at least one protein associated with development of multi-drug resistance.

9. The composition of claim 8, wherein said at least one protein associated with development of multi-drug resistance is a P-glycoprotein, multidrug resistance-associated protein, or lung resistance-related protein.

10. The composition of claim 8, further comprising a chemotherapy drug.

11. The composition of claim 8, wherein said at least one protein is a P-glycoprotein; and the xanthine compound has a structure characterized by formula II, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl,
wherein R¹, R², R³ and R⁴ are not all concurrently hydrogen.

12. The composition of claim 11, wherein the xanthine compound is selected from the group consisting of theophylline, theobromine, 8-cyclopentyl-1,3-dipropylxanthine (DPCPX), 3,7-dimethyl-1-propargylxanthine (DMPX), and 8-(3-chlorostyryl)caffeine (CSC).

13. The composition of claim 11, further comprising a chemotherapy drug.

14. The composition of claim 1, further comprising a pharmaceutically acceptable carrier.

15. A method of treating a patient having a disease or condition associated with expression of ABCG2, comprising administering to the patient a therapeutically effective amount of a composition comprising a pharmaceutically active agent and a xanthine compound, wherein the pharmaceutically active agent is an ABCG2 substrate and the xanthine compound has a structure according to formula (II): 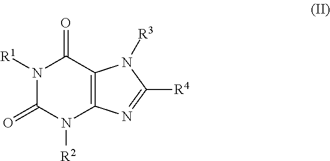
wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, C₂-C₆ alkenyl, alkynyl, aryl, arylalkyl, and arylalkenyl, wherein the aryl or aryl part of the arylalkyl and arylalkenyl is optionally substituted by one to five substituents independently selected from the group consisting of halogen, C₁-C₆ alkyl, hydroxyl and C₁-C₆ alkoxy,
with the provisos that the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, or phenylpropanolamine, when the xanthine compound is caffeine, and
that the xanthine compound is not a component of coffee, tea, or a caffeinated soft beverage.

16. The method of claim 15, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl.

17. The method of claim 15, wherein the xanthine compound is selected from the group consisting of consisting of theobromine, 8-cyclopentyl-1,3-dipropylxanthine (DPCPX), 3,7-dimethyl-1-propargylxanthine (DMPX), and 8-(3-chlorostyryl)caffeine (CSC).

18. The method of claim 15, wherein the disease is a cancer.

19. The method of claim 18, wherein the cancer is a multi-drug resistant cancer characterized by cancerous cells expressing ABCG2.

20. The method of claim 15, wherein the composition is administered orally, intravenously, intraperitoneal, intraarterially, intramuscularly, intracolonically, intracranially, intra-thecally, intraventricularly, intraurethrally, intravaginally, subcutaneously, intraocularly, intranasally, topically, or by any combinations thereof.

21. The method of claim 18, wherein the cancer is selected from the group consisting of brain cancer, lung cancer, stomach cancer, duodenal cancer, esophagus cancer, breast cancer, colon and rectal cancer, bladder cancer, kidney cancer, pancreatic cancer, prostate cancer, ovarian cancer, mouth cancer, eye cancer, thyroid cancer, urethral cancer, vaginal cancer, neck cancer, lymphoma, acute lymphocytic leukemia, chronic myelogenous leukemia, chronic lymphocytic leukemia, hairy cell leukemia and myelomas.

22. A method of improving bioavailability of a pharmaceutically active agent delivered across an ABCG2 expressing membrane to a patient in need thereof, comprising administering to the patient the pharmaceutically active agent in combination with a xanthine compound according to formula II: 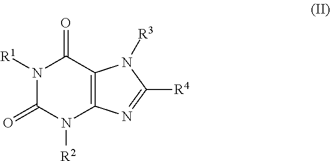
wherein:
R¹ is hydrogen, C₁-C₆ alkyl, C₂-C₆ alkenyl, or C₂-C₆ alkynyl;
R² is hydrogen or C₁-C₆ alkyl;
R³ is hydrogen or C₁-C₆ alkyl optionally substituted by one to three substituents independently selected from hydroxyl and halogen; and
R⁴ is selected from the group consisting of hydrogen, C₁-C₆ alkyl, C₃-C₆ cycloalkyl, C₂-C₆ alkenyl, C₂-C₆ alkynyl, aryl, arylalkyl, and arylalkenyl, wherein the aryl or aryl part of the arylalkyl and arylalkenyl is optionally substituted by one to five substituents independently selected from the group consisting of halogen, C₁-C₆ alkyl, hydroxyl and C₁-C₆ alkoxy,
wherein the pharmaceutically active agent is an ABCG2 substrate,
with the provisos that the pharmaceutically active agent is not ergotamine tartrate, acetaminophen, ibuprophen, Isometheptene Mucate, acetylsalicylic acid or a salt thereof, butalbital, Propoxyphene, Pyrilamine maleate, chlorpheniramine, or phenylpropanolamine, when the xanthine compound is caffeine, and
that the xanthine compound is not a component of coffee, tea, or a caffeinated soft beverage.

23. The method of claim 22, wherein the xanthine compound is administered prior to, simultaneously with, or after the pharmaceutically active agent, with the proviso that the xanthine compound is not administered in the form of coffee, tea, or a caffeinated soft beverage.

24. The method of claim 22, wherein:
R¹ is hydrogen, methyl, 1-propyl, or propargyl;
R² is hydrogen, methyl, or 1-propyl;
R³ is hydrogen, methyl, or 2,3-dihydroxyl-1-propyl; and
R⁴ is hydrogen, cyclopentyl, or 3-chlorostyryl.

25. The method of claim 22, wherein the xanthine compound is selected from the group consisting of consisting of theophylline, theobromine, 8-cyclopentyl-1,3-dipropylxanthine (DPCPX), 3,7-dimethyl-1-propargylxanthine (DMPX), and 8-(3-chlorostyryl)caffeine (CSC).

26. The method of claim 22, wherein the patient is inflicted with a cancer.

27. The method of claim 26, wherein the cancer is a multi-drug resistant cancer characterized by cancerous cells expressing ABCG2.
